(19) United States
(12) Patent Application Publication
(10) Pub. No.: US 2015/0080814 A1
(43) Pub. Date: Mar. 19, 2015

(54) MULTI-LUMEN MULTI-CLAMP LUER LOCK SYSTEM

(71) Applicant: Bio Health Frontiers, Incorporated, Las Vegas, NV (US)

(72) Inventors: Paul Lambert, El Dorado Hills, CA (US); Carlos Gutierrez, El Dorado Hills, CA (US)

(73) Assignee: Bio Health Frontiers, Incorporated

(21) Appl. No.: 14/305,398

(22) Filed: Jun. 16, 2014

Related U.S. Application Data
(60) Provisional application No. 61/835,498, filed on Jun. 14, 2013.

(51) Int. Cl. A61M 39/10; A61M 39/28

(52) U.S. Cl. CPC A61M 39/105; A61M 39/28; A61M 39/1011
USPC 604/250; 604/535

(57) ABSTRACT

An embodiment of the present invention comprises an adapter configured to divide a single input tubular line into multiple tubular lines used in infusion therapies, which has a manifold at one end that comprises a single input port (e.g., female luer, male luer, barbed tubular pole, or straight tubular port) to which a fluidic source may be connected, multiple output ports (e.g., female tubing ports or barbed tubular poles) to which output infusion tubular lines may be connected, a flow disburser feature to ensure even distribution of fluidic flow within the adapter, recessed slots in the sides of the adapter to clamp the output infusion tubular lines without the need for external clamps, and identifying markings on the outside of the adapter to uniquely identify each of the output tubular lines. The adapter may also incorporate an embedded flow restrictor or filter, eliminating the need for external components and their associated connectors.

### CROSS-REFERENCES TO RELATED APPLICATIONS

The present application claims priority from copending Provisional Patent Application No. 61/835,498, filed on 14 Jun. 2013 and entitled “MULTI-LUMEN MULTI-CLAMP LUER LOCK SYSTEM”, the foregoing application being incorporated herein, by reference, in its entirety.

### BACKGROUND OF THE INVENTION

I. Field of the Invention

The present invention relates to the field of medical devices used in infusion and similar therapies, wherein medicinal or other fluidic treatments are conducted via one or more tubular lines from a fluidic source to a patient. In such medical devices, the tubular lines are interconnected via various connector assemblies such as luer connectors and other components. The tubular lines ultimately terminate at treatment areas within a patient via one or more needles or catheters inserted in the patient, which administer the fluidic treatments subcutaneously, intramuscularly, intravenously, epidurally/spinally or by a similar method. Infusion therapy sets customarily use a clamp (typically a roller, slide or pinch clamp) to stop as desired the flow of fluidic treatment through a tubular line leading into the patient's body. These clamps are positioned on and attached to the tubing and are able to slide up and down a certain segment of the tubing. In conjunction with clamps, flow restrictors that limit the inner diameter of the tubing may be inserted in the tubular line to control the rate of flow through the line, and filters that block particulates within the tubular line may be inserted as well.

II. Description of the Related Art

When fluidic therapy is delivered to a patient, a fluidic source and the means to deliver the fluidic treatment to the patient are required. Fluids may be sourced by a syringe and pump, a hanging bag, or other fluidic source. A tubing set comprised of one or more tubular lines connects the fluid source (i.e., the distal end) to the patient (i.e., the proximal end). At the distal end, tubular lines are terminated by connectors (usually female luer connectors) to permit their connection to the source of the fluid. Each tubular fluidic line may include a clamp positioned and engaged in a tubular line to occlude the fluidic flow within the line, or an inline filter to regulate particulate quality. A tubing set may also include an inline flow restrictor to regulate the flow rate within the tubular lines. At the proximal end of the tubular lines, needles (such as subcutaneous, intramuscular, intravenous, epidural/spinal or similar types) deliver the fluidic treatment to the patient's treatment areas.

A feature of infusion therapy is to deliver the fluidic treatment in a distributed fashion via multiple tubular lines to multiple treatment locations on the patient. Typically, a tubular line segment is a single line. Each of these tubular lines can be straight or coiled. In special clinical situations, bifurcated, trifurcated or multi-furcated lines are required. Various line splitting components exist to accomplish the bifurcation, trifurcation, or multiple line splitting of tubular lines to satisfy the requirements. Generally, the distal end of a line splitting component comprises an input port configured with a female luer, while in the proximal direction the component incorporates branches to split one or more times to achieve multiple tubing configurations terminating at the proximal end in multiple connectors (typically male luer connectors). These male luer connectors mate with adjoining connectors affixed to the tubular lines of the tubing set leading to the patient. The utilization of multiple line splitting components and the requisite female/male luer connector pairs increases the total component count, cost and complexity of managing the tubing set, particularly when many tubular lines (or multiple tubing sets) are used.

The splitting of multiple tubular lines has clinical advantages, such as permitting the delivery of the fluid to multiple treatment areas on the patient. However, the assembly of these multiple components often results in asymmetrical configurations of tubing lengths and sub-optimal or unequal flow in each of the multiple fluidic paths, which results in uneven distribution of medication to the patient's treatment areas.

One or more tubular lines in the tubing set may interpose a flow restrictor within the tubular line and in series with other components in the tubular line. Infusion therapy typically use flow restrictors to control the rate of fluidic flow within the tubular lines. The present state of the art interposes the flow restrictor in the tubular line by configuring a female and male luer connector at the ends of a break in the tubular line, then inserting the flow restrictor component in series between the female and male luer connectors. Inserting flow restrictors with the requisite female/male connector luer pairs contributes to the increased total component count, cost and complexity of the tubing set or to sub-optimal ability to handle and manipulate the tubing set for clinical purposes.

One or more tubular lines in the tubing set may also interpose an inline filter to ensure purity from particulates. The present state of the art interposes the filter in the tubular line by configuring a female and male luer connector at the ends of a break in the tubular line, then inserting a filter component in series between the female and male luer connectors. Similarly to adding flow restrictors, inserting filters with the requisite female/male luer connector pairs increases the total component count, cost and complexity of the tubing set.

There exist various products to implement an infusion therapy set consisting of multiple tubular lines; for example, various Carefusion Corporation infusion therapy extension sets such as the MP9264-C*, a “minibore tri-fuse extensions set, 2 MaxPlus clear needleless connectors, 0.2 micron filter, 3 non-removable slide clamps, male spin lock,” as shown in FIG. 10.

Examples of tube-to-tube connectors include various products from ValuePlastics, Inc., such as T210 (-1 -2 -6005 -9 -J1A)*, a “tee connector with 200 series barbs, 1/16″ (1.6 mm) ID tubing,” Y210 (-1 -2 -6005 -9 -J1A)*, a “y connector with 200 series barbs, 1/16″ (1.6 mm) ID tubing,” 3 PF210 (-1 -6005)*, a “three-port F style manifold with 200 series barbs for 1/16″ (1.6 mm) ID tubing,” as shown in FIGS. 11A, 11B, and 11C, respectively.

There exist various products that are examples of clamps and flow restrictors; for example, Carefusion Corporation MFS104*, an “extension set, flow regulator, non-removable slide clamp, male spin lock,” as shown in FIG. 12. There exist various products that are examples of filters; for example, Carefusion Corporation 10013902*, a “low sorbing extension set, 0.2 micron (low-protein binding) filter, 1 needle free-valve, 2-piece male luer lock,” as shown in FIG. 13. These products necessitate assembling various lengths of multi-channel lines with discrete clamps, flow restrictors, filters, and corresponding connector pairs.

### BRIEF SUMMARY OF THE INVENTION

The present invention may address one or more of the problems and deficiencies of the prior art discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

Certain embodiments of the present invention include, but are not limited to, an adapter configured to divide a single input tubular line into multiple tubular lines (“Adapter”), which lead to a patient's treatment areas in infusion therapies or similar uses. The distal end (i.e., facing away from a patient) of the Adapter contains a manifold comprising a single input port or connector (e.g., a female luer connector or a male luer connector) and a proximal end (i.e., facing towards the patient), which contains multiple output ports or connectors (e.g., female tubing ports). The Adapter simplifies the network of multiple tubular lines in a tubing set and reduces the total number of components involved in administering infusion therapy to the patient. This configuration also optimizes distribution of flow through the various tubular lines.

On the distal end of the Adapter, the input connector (e.g., a female luer connector) is mated to its corresponding connector (e.g., a corresponding male luer connector), which may be connected to a fluidic reservoir, or to an input infusion line segment that is connected to a fluidic source.

On the proximal end of the Adapter, the output connectors are mated to output infusion lines directed towards the patient. The joint is typically accomplished by friction, barbed tubular pole connection, or bonding agent or other method of joinder as is known in the art. The multiple output infusion lines terminate in infusion connectors such as luer connectors, adapters, in-line connectors, valves, filters, needles and other infusion delivery mechanisms, or in open-ended tubing for other connectivity.

On the proximal end of the Adapter is fashioned a collar that encircles the output connectors. Within each side of the collar adjacent to each output connector, a recessed slot is configured in the collar that extends a certain depth from the bottom edge of the collar. When a section of the infusion line is inserted and clamped into its respective slot, the infusion line is pinched off, the fluidic flow within is occluded, and the slot acts as a clamp. The use of slots replaces the need for additional discrete clamps and simplifies the ability of the clinician to close off one or more tubular lines.

An alternative embodiment of the Adapter incorporates an embedded flow restrictor configured within the Adapter, inserted in-line between the input connector and the output connectors. Integrating the flow restrictor within the Adapter eliminates an additional external component as well as an associated male/female connector pair, thereby reducing the complexity of managing tubular lines inherent to infusion therapies, particularly when multiple tubing sets and tubular lines are used.

An alternative embodiment of the Adapter incorporates an embedded filter configured within the Adapter, inserted in-line between the input connector and the output connectors. Integrating the filter within the Adapter eliminates an additional external component as well as an associated male/female connector pair, thereby reducing the complexity of managing tubular lines inherent to infusion therapies, particularly when multiple tubing sets and tubular lines are used.

### BRIEF DESCRIPTION OF THE DRAWINGS

FIG. 1 Elevation side view of adapter

FIG. 2 Side view of adapter

FIG. 3 Cutaway sectional side view of adapter at Section D-D

FIG. 4 Cross sectional backwards facing view of adapter at Section G-G

FIG. 5 Cross sectional backwards facing view of alternative embodiment of adapter at Section G-G

FIG. 6 Cross sectional backwards facing view of adapter at Section F-F

FIG. 7 Frontwards facing elevation view of adapter from distal end of adapter

FIG. 8 Elevation exploded side view of adapter with embedded flow restrictor and embedded filter

FIG. 9 Elevation exploded side view of adapter showing fluidic source, adapter, input tubular line and output tubular lines

FIG. 10 Plan view of multiple tubular lines splitter according to the prior art

FIGS. 11A, 11B and 11C Plan view of three tube-to-tube connectors according to the prior art

FIG. 12 Plan view of an exemplary flow regulator according to the prior art

FIG. 13 Plan view of an inline filter according to the prior art.

The following is a list of reference numerals appearing in the drawing:
1 Adapter
2 Collar
3 Support strut
4 Proximal opening of collar
5 Output connector
6 Slot
7 Identifiers
8 Base of input connector
9 Input connector
10 Ridge of input connector
11 Distal opening of input connector
12 Flow disburser
13 Flow restrictor
14 Filter
15 Fluid source bag or syringe
16 Fluid source clamp
17 Upper tubing line segment
18 Male luer connection
19 Lower tubing line segments

### DETAILED DESCRIPTION OF THE INVENTION

In the following description, for purposes of explanation, numerous specific details are set forth in order to provide an understanding of various embodiments of the inventive subject matter. It will be evident, however, to those skilled in the art that embodiments of the inventive subject matter may be practiced without these specific details. In general, well-known structures and techniques have not been shown in detail.

As used herein, the term “or” may be construed in either an inclusive or exclusive sense. Similarly, the term “exemplary” is construed merely to mean an example of something or an exemplar and not necessarily a preferred or ideal means of accomplishing a goal. Additionally, although various exemplary embodiments discussed below focus on verification of experts, the embodiments are given merely for clarity and disclosure. Alternative embodiments may employ other systems and methods and are considered as being within the scope of the present invention.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment.

In the description that follows, any reference to either orientation or direction is intended primarily and solely for the purpose of illustration and is not intended in any way as a limitation of the scope of the present invention or its claims. Also, the particular embodiments described herein although being noted as preferred are not to be considered as limiting of the present invention. Furthermore, like-parts or like-elements in the various drawings hereto are identified by like-numerals.

### Adapter Elevation View

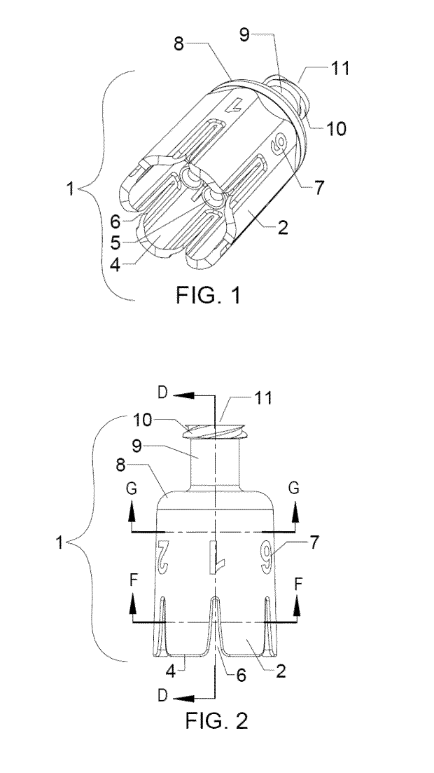

Certain embodiments of the present invention include, but are not limited to, a device according to the present invention is illustrated in FIG. 1 as an elevation side view of a single-input to multi-output Adapter 1. The Adapter 1 is molded out of high-impact plastic or lightweight metal as are known in the art.

### Collar and Connectors

In FIG. 1, at the proximal end of the Adapter 1 is fashioned a collar 2 (“Collar”) that encircles the proximal opening 4 of the Adapter 1. The preferred embodiment of the Collar 2 is configured in a circular shape, but alternatively the Collar 2 may be oval shaped or have a multi-faceted surface, such as hexagonally, square, rectangular, or another contiguous shape. In the preferred embodiment of the Adapter 1, within the Collar 2 are configured six output connectors 5 (“Output Connectors”) to accommodate a tubing set of six tubular lines. Other contemplated embodiments include two, four, eight, ten, twelve, or any reasonable number of Output Connectors 5 that can be configured to fit within the Collar 2 comprising a shape as described above.

### Clamps

In FIG. 1, in the preferred embodiment of the Collar 2, adjacent to each Output Connector 5 is fashioned a slot 6 that extends from the proximal opening 4 of the Collar 2 for a certain depth in the distal direction. As will be discussed below, the width of each Slot 6 is configured to taper slightly according to industry standards for tubing occlusion of a given range of outer diameters of infusion lines, so that the infusion line may be bent into and clamped within the Slot 6 and thereby act as a clamp to stop any fluidic flow within the line.

### Numbering

In FIG. 1, on the outside surface of the preferred embodiment of the Collar 2 are fashioned numerals 7 (“Identifiers”). Other contemplated embodiments of the Identifiers 7 include identifying markings such as letters, symbols, colors, tactile impressions or projections, or tactile dots configured as Braille symbols. Clinician confusion may be reduced by incorporating markings or symbols at the terminating ends of the infusion tubular lines (i.e., at the patient treatment area) that match the Identifiers 7 configured on the Adapter 1.

### Input Connector

In FIG. 1, at the distal end of the preferred embodiment of the Adapter 1 is fashioned an input connector (“Input Connector”) 9 that extends in the distal direction from the base of the input connector 8. Also apparent in this figure is the screw ridge 10 of the female connector formed in the Input Connector 9, which is typical for a luer female connector that mates with an adjoining male connector attached to an input tubular line. Alternative embodiments for the Input Connector 9 are contemplated, such as a male luer connector, a tubular section, a barbed tubular pole connection, a tapered orifice, or other connectors as are known in the art, or simply a port to permit interconnection with other components known in the art. FIG. 1 also shows the position of the distal opening 11 of the Input Connector 9 for reference.

### Adapter Side View

FIG. 2 illustrates a side view of an embodiment of Adapter 1, showing the Collar 2, the position of the Collar's proximal opening 4, the Slots 6, and the Identifiers 7. FIG. 2 shows the depth of the preferred embodiment of each Slot 6 extending from the proximal opening 4 (“Collar Opening”) of the Collar 2 in the distal direction.

FIG. 2 illustrates the Input Connector 9 and its base 8, and the position of the distal opening 11 of the Input Connector 9 for reference. Also shown is the screw ridge 10 (“Ridge”) at the distal edge of the Input Connector 9, which is typical for industry standard female luer connectors.

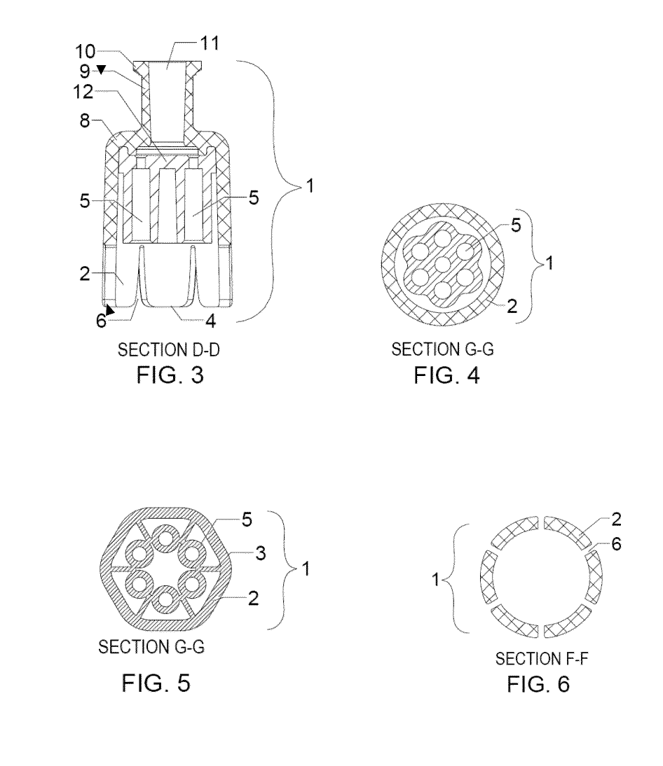

FIG. 3 illustrates a cutaway sectional side view of an embodiment of Adapter 1 at Section D-D, showing the Collar 2, the proximal Collar Opening 4, two Output Connectors 5, the Slots 6, the base 8 of the distal Input Connector 9, the distal Input Connector 9, and the Ridge feature 10 of a female luer connector.

FIG. 3 also shows the distal opening 11 of the Input Connector 9 and an internal pathway extending from the distal opening 11. At the bottom of the internal pathway is a rounded button that acts as a flow disburser (“Flow Disburser”) 12, which presents a smooth profile and contour to the fluidic flow passing through the distal opening 11 and its internal pathway within the Input Connector 9, thereby ensuring an even distribution of fluidic flow to all lumens of the Output Connectors 5 and optimizing distribution of fluid, particularly when compared with the present state of the art.

In FIG. 3, the width of an embodiment of each Slot 6 is configured to taper slightly from a flared width to a slightly smaller width in the distal direction (as per the industry standard specification corresponding to a given range of infusion tube outer diameters). When a segment of tubular line is bent into and clamped within the Slot 6, the slot acts as a clamp to stop any fluidic flow within the tubular line. Alternative embodiments of these clamping Slots 6 are contemplated where one or more additional slots may be present, corresponding to one infusion line connected to an Output Connector 5, where one or more of the additional slots are configured with a differing depth and width to provide intermediate positions of clamping closure, thereby affording additional degrees of flow restriction.

### Input Connector

In FIG. 3, an embodiment of the Input Connector 9 and Ridge 10 is configured as a female luer connector. Alternative embodiments are contemplated where the connector is configured as a male luer connector, as a barbed tubular post connection configured with barbs on the external surface of the tubular post, as is known in the art, to which a bare tube is attached via friction against the barb of the input connector 9 without the requirement of an associated connector, or as a straight tubular post. The diameter of the Input Connector opening 11 (“Input Connector Opening”) is configured to match the corresponding connector and outer diameter of its attached tubular line.

### Adapter Backwards Facing Views

FIG. 4 illustrates a backwards-facing cross-sectional view of an embodiment of Adapter 1 at Section G-G. FIG. 4 shows the Collar 2 and Output Connectors 5. Each Output Connector 5 is placed adjacently to the inner surface of the Collar 2 and positioned equidistant from the center point of the Adapter 1.

### Output Connector

In FIG. 4, the inner diameter of an embodiment of each Output Connector 5 is configured to match the outer diameter of corresponding tubular line that would be inserted into, affixed with a bonding agent or otherwise connected with the Output Connector 5.

An alternative embodiment of each Output Connector 5 comprises a barbed tubular pole connection configured with barbs on the outer surface of the tubular post, as is known in the art, to which a corresponding tubular line is inserted and attached to each of the output connectors 5 via friction against the barb of each output connector 5 without the requirement of a bonding agent.

FIG. 5 illustrates a backwards-facing cross-sectional view of an alternative embodiment of Adapter 1 at Section G-G, which includes a support structure 3. In this alternative embodiment, the support structure 3 extends from the inner wall of the Collar 2 to the outer wall of the nearest Output Connector 5 to provide additional strength and stability.

FIG. 6 illustrates a backwards-facing cross-sectional view of an embodiment of Adapter 1 at Section F-F. FIG. 6 shows the Collar 2 and Slots 6.

### Frontwards Facing Elevation View

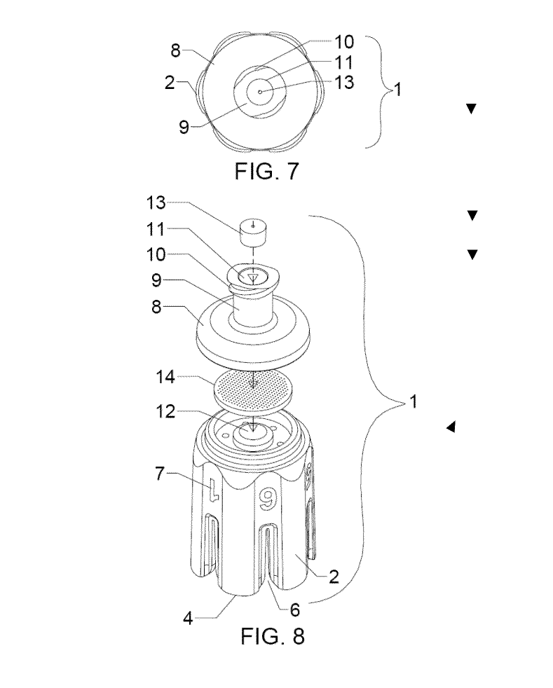

FIG. 7 illustrates a frontwards-facing elevation view of an embodiment of Adapter 1 from the distal end of the Adapter 1. FIG. 7 shows the top of the Input Connector 9 (including a portion of the threading at the outer edge of the Ridge 10 and the inner diameter of its distal opening 11 of the female luer lock. FIG. 7 shows the inner diameter of an inserted Flow Restrictor (defined below) 13. FIG. 7 also shows the Adapter's circular base 8 and the edges of a multi-facet shaped Collar 2.

### Flow Restrictors and Filters

FIG. 8 illustrates an elevation exploded side view of an alternative embodiment of the Adapter 1, showing the Collar 2, proximal Collar Opening 4, Slots 6, Identifiers 7, Flow Disburser 12, base of the input connector 8, Input Connector 9, Ridge 10, and distal opening of the input connector 11. This figure also shows an optional embedded flow restrictor (“Flow Restrictor”) 13 that may be incorporated into the Adapter 1 within the Input Connector 9, and/or an optional embedded filter (“Filter”) 14 that may be incorporated into the Adapter 1 within the base 8 of the Input Connector 9. Alternative embodiments are also contemplated where the Flow Restrictor 13 and the Filter 14 are placed at other locations within the internal pathways of the Adapter 1.

### Method of Use

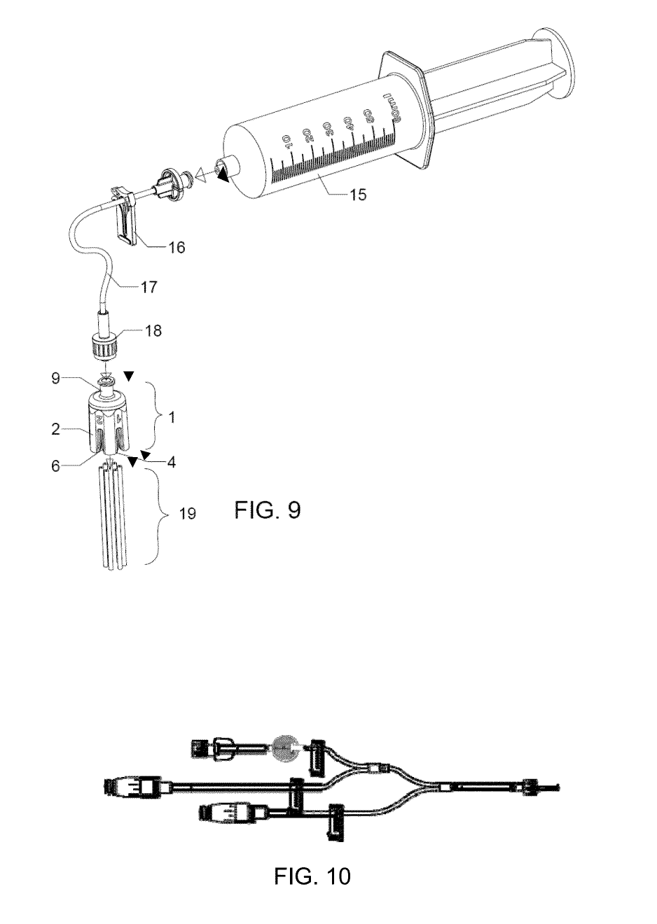

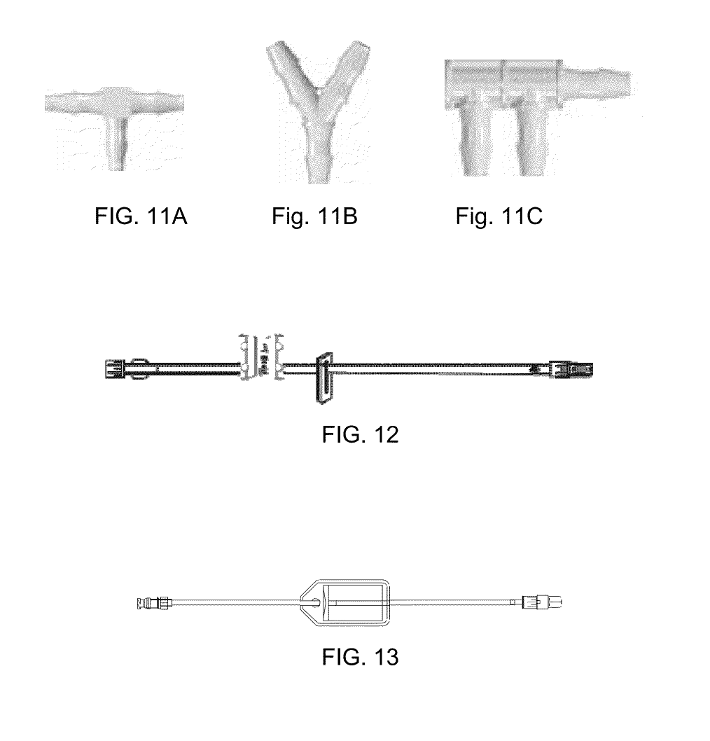

Certain embodiments of the present invention include, but are not limited to, a method of using the Adapter 1. FIG. 9 is referenced in discussing an embodiment of a method of using the Adapter 1. In preparation for an embodiment of a method of using the Adapter 1, a clinician prepares a fluidic reservoir comprised of a syringe pump 15 or other fluidic source (such as a hanging bag of fluid or other source) and connects to an upper tubular line segment 17 that extends from the bottom of the fluidic reservoir 15, as is known in the art. The clinician may attach a master clamp or valve 16 to ensure that no fluid begins dripping through the upper tubular line segment 17. The end of the upper tubular line segment 17 typically terminates in a male luer connector 18. First, in an embodiment of a method of using the Adapter 1, the Adapter 1 is interposed in the tubing set by connecting the Adapter's input connector 9 (a female luer connector) to the male luer connector 18 of the upper tubular line segment 17.

In an alternative embodiment of a method of using the Adapter 1, the Adapter 1 is interposed in the tubing set by connecting the Adapter's input connector 9 (e.g., a female luer connector) directly to a corresponding connector (e.g., a male luer connector) of the fluidic reservoir 15 without the requirement of an intervening tubular line segment and associated connectors.

In another alternative embodiment of a method of using the Adapter 1, the Adapter's input connector 9 comprises a barbed tubular post configured with barbs on the outer surface of the tubular post, as is known in the art, rather than a female luer connector, to which a bare tube is attached via friction against the barb of the input connector 9 without the requirement of an associated connector.

Second, in an embodiment of a method of using the Adapter 1, each of the six output tubular lines 19 is affixed to each of the six output connectors 5 (not in view) within the proximal opening 4 of the Collar 2 using a bonding agent as is known in the art. In an alternative embodiment, it is contemplated that each of the output connectors 5 (not in view) is fashioned with a barbed tubular post configured with barbs on the outer surface of the tubular post, as is known in the art, so that each of the output tubular lines 19 is inserted and attached into each of the output connectors 5 via friction against the barb of each output connector 5 without the requirement of a bonding agent.

Third, in an embodiment of a method of using an alternative embodiment of the Adapter 1, the clinician may select an Adapter 1 that is configured with an embedded flow restrictor 13 (not in view) and/or an embedded filter 14 (not in view) according to the clinician's requirement.

Fourth, in an embodiment of a method of using the Adapter 1, to occlude fluidic flow within each of the output tubular lines 19 as the clinician desires, before initiating the flow from the fluidic source by engaging the syringe driver or pump or by releasing a master clamp or valve 16 at the bottom of the fluidic source 15, the clinician pulls a segment of each of the output tubular lines 19 through its respective Slot 6 in the Adapter 1, thereby clamping each output tubular line 19 as desired within the Adapter 1.

Fifth, the clinician primes all tubing sets to purge air out, as is known in the art.

Sixth, the clinician attaches the proximal ends of all the output tubular lines 19 to the patient at the desired treatment areas to administer infusion therapy as per procedures as known in the art.

Seventh, when the clinician is ready to begin the infusion therapy, the clinician unclamps the output tubular lines 19 by pulling each segment of output tubular line 19 out of its respective Slot 6. If the clinician desires to later stop the flow of infusion therapy, the clinician clamps an output tubular line 19 by pulling a segment of the output tubular line 19 back into its respective Slot 6 in the Adapter 1. In the case in which a tubing segment 17 is used, the master clamp or valve 16 can be alternatively used to close or open the fluidic flow within all output tubular lines 19 simultaneously.

*TM and brands are the property of their respective owners.

I claim:

1. A device, comprising:
a housing, comprising a closed distal end and an open proximal end;
an input connector providing an opening through the closed distal end of said housing;
an internal pathway connected to said input connector;
an at least one output lumen connected to said internal pathway; and
an at least one output connector positioned within the open end of said housing, said at least one output connector connected to said at least one output lumen.

2. A device as in claim 1, further comprising:
said housing formed in a substantially circular shape about the internal axis running between the distal and proximal ends.

3. A device as in claim 1, further comprising:
an at least one occlusion feature fashioned in the surface of said housing at the edge of the proximal end of said housing extending in the distal direction parallel to the axis running between the distal and proximal ends of said housing.

4. A device as in claim 2, further comprising:
an at least one occlusion feature fashioned in the surface of said housing at the edge of the proximal end of said housing extending in the distal direction parallel to the axis running between the distal and proximal ends of said housing.

5. A device as in claim 1, further comprising:
a flow restrictor embedded inline said internal pathway between said input connector and said at least one output lumen.

6. A device as in claim 1, further comprising:
a filter embedded inline said internal pathway between said input connector and said at least one output lumen.

7. A device as in claim 1, further comprising:
a flow restrictor embedded inline said internal pathway between said input connector and said at least one output lumen; and
a filter embedded inline said internal pathway between said input connector and said at least one output lumen.

8. A method of preparing infusion lines, comprising:
using the device of claim 1;
connecting a fluidic source and tubing to the device's input connector;
connecting an at least one output tubular line to each at least one output connector;
occluding the fluidic flow within an at least one output tubular line by pulling a segment said at least one output tubular line in an at least one occlusion feature; and
unclamping said at least one output tubular line by pulling said segment of said at least one output tubular line out of said at least one occlusion feature.
